SEQUENCE LISTING (10 sequences)

SEQ ID NO: 1 - protein, 4 aa, Artificial Sequence
  Description of Artificial Sequence Synthetic peptide
GRGD

SEQ ID NO: 2 - DNA, 21 bp, Artificial Sequence
  Description of Artificial Sequence Synthetic primer
acaagctccacctggactaca

SEQ ID NO: 3 - DNA, 19 bp, Artificial Sequence
  Description of Artificial Sequence Synthetic primer
tcttcttcacacgcagttt

SEQ ID NO: 4 - DNA, 20 bp, Artificial Sequence
  Description of Artificial Sequence Synthetic primer
gacggccgaggtgatagctt

SEQ ID NO: 5 - DNA, 21 bp, Artificial Sequence
  Description of Artificial Sequence Synthetic primer
catggctggtcttcccgttgc

SEQ ID NO: 6 - DNA, 16 bp, Artificial Sequence
  Description of Artificial Sequence Synthetic primer
aaagcccagcgactct

SEQ ID NO: 7 - DNA, 21 bp, Artificial Sequence
  Description of Artificial Sequence Synthetic primer
ctaaacggtggtgccatagat

SEQ ID NO: 8 - DNA, 20 bp, Artificial Sequence
  Description of Artificial Sequence Synthetic primer
cgacctggaagtccaactac

SEQ ID NO: 9 - DNA, 18 bp, Artificial Sequence
  Description of Artificial Sequence Synthetic primer
atctgctgcatctgcttg

SEQ ID NO: 10 - protein, 5 aa, Artificial Sequence
  Description of Artificial Sequence Synthetic peptide
YIGSR

(19) United States
(12) Patent Application Publication
(10) Pub. No.: US 2012/0226295 A1
(43) Pub. Date: Sep. 6, 2012

(54) Fiber-Reinforced Laminated Hydrogel / Hydroxyapatite Nanocomposites

(75) Inventor: Esmaiel Jabbari, Columbia, SC (US)

(73) Assignee: UNIVERSITY OF SOUTH CAROLINA, Columbia, SC (US)

(21) Appl. No.: 12/820,701

(22) Filed: Jun. 22, 2010

Related U.S. Application Data
(60) Provisional application No. 61/269,223, filed on Jun. 22, 2009.

(51) Int. Cl. A61L 27/52; B29C 70/18; B82Y 40/00

(52) U.S. Cl. 606/151; 156/324; 977/961

(57) ABSTRACT

In accordance with certain embodiments of the present disclosure, a method for forming a laminated nanocomposite is provided. The method includes applying a hydrogel precursor solution to a first layer of poly(L-lactide) nanofiber mesh. A second layer of poly(L-lactide) nanofiber mesh is stacked on the first layer with at least a portion of the hydrogel precursor solution being situated between the first layer and the second layer. The method further includes compressing the first layer and second layer together wherein the first layer and second layer are crosslinked to one another by the hydrogel precursor solution to form a laminated nanocomposite. Furthermore, the laminate layers, prior to crosslinking, can be wrapped around a rod and crosslinked to form a laminated tubular nanocomposite.

### CROSS-REFERENCE TO RELATED APPLICATION

The present application is based on and claims priority to U.S. Provisional Application 61/269,223 having a filing date of Jun. 22, 2009, which is incorporated by reference herein.

### BACKGROUND

There are more than 0.5 million skeletal injuries in the United States annually that require bone graft procedures to ensure rapid skeletal repair. These include bone loss after skeletal trauma, resection of tumors, voids following osteoporotic fractures, and maxillofacial defects. Novel biomaterials that can provide temporary structural support to the regenerating region, initiate the cascade of osteogenesis and mineralized matrix formation, and degrade concurrent with the production of ECM are urgently needed for limb, head, and face reconstruction of patients with multiple traumatic injuries. For example, as a scaffold, the biomaterial could be used in patients who have undergone frontotemporal craniotomy, with severely resorbed maxilla, as resorbable trays in reconstruction of large mandibular defects, and in alveolar ridge augmentation.

The porous collagen sponge is the most widely used scaffold by orthopedic surgeons because it provides an osteoconductive matrix for migration of bone marrow stromal (BMS) cells and a template for mineralization. However, additional mechanical protection in the form of a metallic cage is required to prevent deformation of the scaffold due to soft tissue compression. Bioactive calcium phosphate (CaP) ceramic scaffolds are used clinically in spine fusion but, due to low initial strength, their use is limited to defects that are subject to uniform loading. To improve bending strength and compressive modulus, composites of CaP with poly(L-lactide) (L-PLA), poly(D,L-lactide), and poly(lactide-co-glycolide) (PLGA) have been developed. Although PLGA/hydroxyapatite (HA) composites provide some structural support during bone repair, the major drawback is the hydrophobicity of PLGA polymers. Unlike the collagenous phase in the natural bone, PLGA can not support complex cell-matrix and cell-cell interactions, solubilization of proteins, and growth factor modulation required for osteogenesis and vasculogenesis which results in undesirably long implantation periods for defect repair. Composites of bioactive ceramics with natural hydrogels like collagen type I promote bone formation and certain compositions harden in-situ but they are prone to fatigue fracture. Nutrients and oxygen diffuse readily through synthetic and natural hydrogels and their hydrophilic structure supports complex cell-cell and cell-matrix interactions.

In view of the above, a need exists for biomaterials that can provide temporary structural support to the regenerating region, initiate the cascade of osteogenesis and mineralized matrix formation, and degrade concurrent with the production of ECM for limb, head, and face reconstruction of patients with multiple traumatic injuries.

### SUMMARY

In accordance with certain embodiments of the present disclosure, a method for forming a laminated nanocomposite is provided. The method includes applying a hydrogel precursor solution to a first layer of nanofiber mesh, the nanofiber mesh including a biocompatible synthetic polymer. A second layer of nanofiber mesh is stacked on the first layer with at least a portion of the hydrogel precursor solution being situated between the first layer and the second layer. The method further includes compressing the first layer and second layer together wherein the first layer and second layer are crosslinked to one another by the hydrogel precursor solution to form a laminated nanocomposite.

In still other embodiments of the present disclosure, a laminated nanocomposite is disclosed. The laminated nanocomposite includes a first layer of poly(L-lactide) nanofiber mesh and a second layer of poly(L-lactide) nanofiber mesh stacked on the first layer. The first layer and second layer are compressed together and crosslinked to one another with a hydrogel precursor.

Other features and aspects of the present disclosure are discussed in greater detail below.

### BRIEF DESCRIPTION OF THE DRAWINGS

A full and enabling disclosure, including the best mode thereof, directed to one of ordinary skill in the art, is set forth more particularly in the remainder of the specification, which makes reference to the appended figures in which:

FIG. 1 illustrates a reaction scheme for the synthesis and crosslinking of PLEOF.

FIG. 2 illustrates a schematic diagram for fabrication of a laminated nanocomposite in accordance with certain aspects of the present disclosure.

FIG. 3 illustrates scanning electron microscope images of the fiber mesh (a), one-layer laminate (b), and 4-layer laminate (c). Arrows in FIG. 2c show each layer in the multi-layer laminate.

FIG. 4 illustrates (a) Water uptake (weight of water/dry weight) and (b) Young's moduli under dry and wet conditions of the laminates. Experimental groups include 30/70 PLEOF hydrogel (Gel), PLEOF hydrogel with 10% HA (Gel-HA), L-PLA electrospun fiber mesh (Fiber), L-PLA fiber mesh/PLEOF hydrogel laminate (Lam), laminate with 10% HA in PLEOF (Lam-HA), laminate conjugated with 1% Ac-GRGD in PLEOF (Lam-RGD) and laminate with 1% Ac-GRGD and 10% HA (Lam-RGD-HA). For the wet conditions in (b), samples were soaked in primary medium without FBS for 24 h prior to DMA measurements. The gray area in (a) is the range for water content of the natural bone excluding the mineral component. The gray area in (b) is the reported range for Young's modulus of wet human cancellous bone. Error bars correspond to means±1 SD for n=3.

FIG. 5 illustrates degradation characteristics with incubation time for L-PLA fiber mesh (Fiber), 30/70 PLEOF hydrogel conjugated with 1% RGD (Gel-RGD), laminate of L-PLA with 30/70 PLEOF hydrogel (Lam) and laminate with 10% HA (Lam-HA).

FIG. 6 illustrates a typical SEM image of the BMS cells on the laminate with 10% HA (Lam-HA).

FIG. 7 illustrates DNA content (a), ALPase activity (b) and calcium content (c) of the BMS cell seeded on different samples with incubation time. Groups include Gel-RGD, Fiber, Lam, Lam-HA, Lam-RGD and Lam-RGD-HA. One star indicates a statistically significant difference between the test group and Fiber for the same incubation time. Two stars indicate a statistically significant difference between the test group (Lam-HA, Lam-RGD or Lam-RGD-HA) and the Lam group for the same time. Error bars correspond to means±1 SD for n=3.

FIG. 8 illustrates mRNA expression levels (as fold difference) of OP (a), OC (b) and ON (c) genes of the BMS cells, seeded on different samples and cultured in osteogenic medium, as a function of incubation time. Groups include Gel-RGD, Lam, Lam-HA, Lam-RGD and Lam-RGD-HA. One star indicates a statistically significant difference between the test group and Gel-RGD group for the same incubation time. Two stars indicate a statistically significant difference between the test group (Lam-HA, Lam-RGD or Lam-RGD-HA) and the Lam group for the same time. Three stars indicate a statistically significant difference between the Lam-RGD-HA group and the Lam-HA or Lam-RGD group for the same time. Error bars correspond to means±1 SD for n=3.

FIG. 9 illustrates a PLGA nanofiber mesh (A) is coated with a crosslinkable precursor solution; the coated fibers (B) are stacked together to form a laminate (C); the laminate is wrapped tightly around a cylindrical rod and crosslinked to form a fiber-reinforced laminated tube (D).

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment, can be used on another embodiment to yield a still further embodiment. Thus, it is intended that the present disclosure covers such modifications and variations as come within the scope of the appended claims and their equivalents.

Bone exhibits hierarchical levels of organization from macroscopic to nanoscale. At the microscale, fibrils are glued together by ECM proteins to form laminated structures (osteons) that make bone elastic and allow diffusion of nutrients and oxygen to cells embedded in the bone matrix. Structures and materials that mimic the morphology of bone at the micro-scale have the potential to accelerate bone formation and facilitate bone remodeling. In the present disclosure, a fiber-reinforced laminated hydrogel/calcium phosphate nanocomposite is described which can mimic the laminated structure of osteons in bone. Random and aligned poly(L-lactide) (L-PLA) nanofiber mesh was fabricated by electrospinning However, any suitable biocompatible synthetic polymer can be utilized in connection with the present disclosure including poly(lactide) and poly(glycolide) and their copolymers (PLGA), poly(caprolactone) (PCL) and its copolymers with PLGA, poly(propylene fumarate) and its copolymers with PLGA and PCL, polyhydroxyalkanoate (PHA), copolymers of PLGA with poly(ethylene glycol) (PEG), poly(anhydrides), polydioxanone, poly(trimethylene carbonate), poly(ester amides), poly(ortho esters), poly(amino acids), polyphosphazenes, and polyphosphoesters and combinations thereof.

The fiber mesh can be dipped in a hydrogel precursor solution containing calcium phosphate nanocrystals. The macromer can be a multi-arm (2 or more) copolymer of lactide, glycolide, fumaric acid, and ethylene glycol monomers with controlled hydrophilic/hydrophobic ratio. The number of arms of the macromer depends on the number of functional groups of the initiator. Next, the dipped layers are stacked and pressed against each other, and crosslinked to form a laminated fibrous nanocomposite. Bioactive agents can be functionalized and incorporated into the hydrogel network to promote cell adhesion, migration, differentiation, and maturation of progenitor cells. For instance, growth factors, differentiation factors, or the like can be incorporated into the hydrogel network to control function of seeded cells. In that regard, such seeded cells can be seeded on the laminates or inside the laminated composites. Lamination produces a multi-functional substrate for bone formation. The fibrous component provides dimensional stability while the hydrogel component facilitates diffusion of oxygen, nutrients, and solubilization of growth factors. The apatite nanocrystals enhance compressive modulus and provide an osteoconductive substrate for mineralization.

In accordance with the present disclosure, a bone-mimetic laminated structure is developed to facilitate bone formation. Sheets of L-PLA nanofibers were fabricated by electrospinning The sheets were dipped in a hydrogel/nanoapatite precursor solution, stacked and pressed together, and allowed to crosslink by photopolymerization to form a fiber-reinforced hydrogel/apatite laminated structure. The precursor solution is based on a novel macromer, poly(lactide-co-glycolide-ethylene oxide-fumarate) (PLEOF), that can be crosslinked in aqueous environment with redox or ultraviolet initiators to produce a biodegradable hydrogel. The crosslink density can be adjusted by the initiator concentration and density of fumarate groups on PLEOF chains. The degradability and water content of the hydrogel can be tailored to a particular application by varying the molecular weight of the lactide chains and the ratio of lactide (LA) to poly(ethylene glycol) (PEG) in PLEOF macromer. PLEOF degradation can be modulated to the migration rate of progenitor cells by crosslinking PLEOF with a biologically degradable crosslinker. The modulus of PLEOF/apatite composite can be enhanced by treating the surface of the apatite crystals with an acrylate-functionalized glutamic acid sequence. Bioactive peptides can be bulk-conjugated or grafted to PLEOF to facilitate adhesion and differentiation of BMS cells. For example, integrin-binding Arg-Gly-Asp (RGD) peptide can be functionalized by reaction with acrylic acid to form acrylamide-terminated RGD (Ac-GRGD) and conjugated to PLEOF hydrogel by the reaction between the acrylamide group of Ac-GRGD and PLEOF fumarate groups.

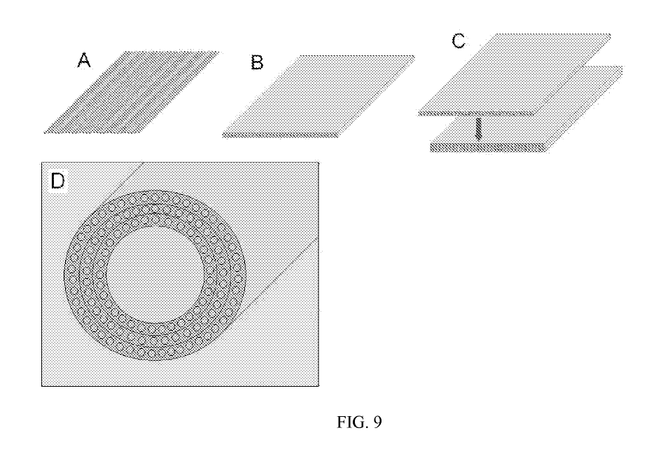

In certain embodiments, laminates in accordance with the present disclosure can be wrapped tightly around a cylindrical rod and crosslinked to form a fiber-reinforced laminated tubular nanocomposite, as illustrated in FIG. 9. In this regard, cells can be seeded inside the laminated tubular nanocomposite.

The present disclosure can be better understood with reference to the following examples.

### EXAMPLES

### Materials

Triethylamine (TEA), tin (II) 2-ethylhexanoate (TOC), N-vinyl-2-pyrrolidone and piperidine were purchased from Sigma-Aldrich (St. Louis, Mo.). Fumaryl chloride (FuCl; Sigma-Aldrich) was purified by distillation and PEG (Sigma-Aldrich; nominal molecular weight of 4.3 kDa) was dried by azeotropic distillation from toluene. Dichloromethane (DCM; Acros Organics) was dried by distillation over calcium hydride (Sigma-Aldrich). Diethylene glycol (DEG), N,N-dimethylformamide (DMF), trifluoroacetic acid (TFA), N,N-dimethylaminopyridine (DMAP), N,N′-diisopropylcarbodiimide (DIC), hydroxybenzotriazole (HOBt), acetonitrile (MeCN), triisopropylsilane (TIPS), N,N′-methylene bisacrylamide (BISAM), ammonium persulfate (APS) and N,N,N′,N′-tetramethylethylenediamine (TMEDA) were purchased from Acros Organics (Fisher; Pittsburg, Pa.). L-lactide monomer (LA; >99.5% Purity by GC) was purchased from Ortec (Easley, S.C.). High-molecular-weight L-PLA (0.9-1.2 dl g-1 intrinsic viscosity and 185 kDa weight average molecular weight) and 1,1,1,3,3,3-hexafluoro-2-propanol (HFIP) were purchased from Durect Corp. (Pelham, Ala.) and VWR (West Chester, Pa.), respectively. The Rink Amide NovaGel™ resin and all Fmoc-protected amino acids were purchased from Novabiochem (EMD Biosciences, San Diego, Calif.). Dulbecco's phosphate-buffer saline (PBS) and Dulbecco's modified Eagle's medium (DMEM; 4.5 g l-1 glucose with L-glutamine and without sodium pyruvate) were purchased from Cellgro (Mediatech; Herndon, Va.).

### Synthesis and Characterization of PLEOF Macromer

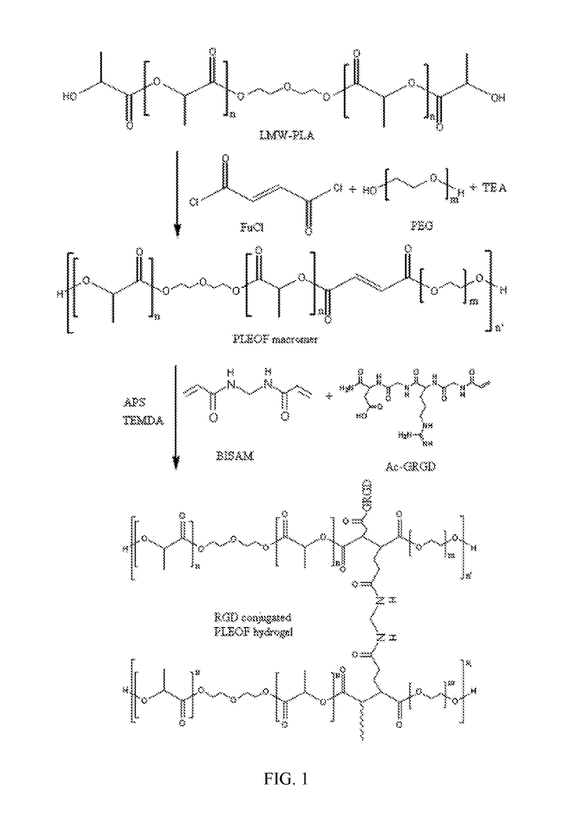

Low-molecular-weight poly(L-lactide) (LMW-PLA) was synthesized by ring opening polymerization of LA monomer as described previously in Jabbari E, He X Z, Synthesis and characterization of bioresorbable in situ crosslinkable ultra low molecular weight poly(lactide) macromer, J Mater Sci Mater Med. 2008; 19(1):311-318 and Jabbari E, He X, Valarmathi M, Sarvestani A, Xu W, Material properties and bone marrow stromal cells response to In situ crosslinkable RGD-functionlized lactide-co-glycolide scaffolds, J Biomed Mater Res A. 2008; 89A(1):124-137, both incorporated by reference herein. DEG and TOC were used as the bifunctional initiator and polymerization catalyst, respectively. PLEOF macromer was synthesized by condensation polymerization of LMW-PLA and PEG with FuCl (see FIG. 1). The weight ratio of PEG to LMW-PLA was 70:30 to produce a hydrophilic water-soluble terpolymer. After completion of the reaction, the product was dissolved in DCM, precipitated twice in ether, dried in vacuum (<5 mm Hg) for at least 12 h and stored at −20° C. until use. Chemical structure of the macromers was characterized by a Varian Mercury-300 1H NMR (Varian, Palo Alto, Calif.). The polymer sample was dissolved in CDCl3 at a concentration of 50 mg ml-1 and 1 vol. % tetramethylsilane (TMS) was used as the internal standard. The molecular weight distribution of the macromers was measured by gel permeation chromatography (GPC). Measurements were carried out with a Waters 717 GPC system (Waters, Milford, Mass.) connected to a model 410 refractive index detector. A 50 μl aliquot of the sample (2 mg ml-1 in THF) was eluted through a styragel HR 4E column (300×7.8 mm, Waters) with degassed THF at a flow rate of 1 ml min-1. Monodisperse polystyrene standards (0.58-66.35 kDa and polydispersities of <1.1, Waters) were used to construct the calibration curve.

### Electrospinning and Characterization of L-PLA Fiber Mesh

The L-PLA fiber mesh was prepared by electrospinning from HFIP solvent (9 wt. %) as described Xu W J, He X Z, Sarvestani A S, Jabbari E, Effect of a low-molecular-weight cross-linkable macromer on electrospinning of poly(lactide-co-glycolide) fibers, J Biomat Sci Polym Ed. 2007; 18(11):1369-1385, incorporated by reference herein. The use of HFIP as the solvent resulted in the production of bead-free fibers at the lowest L-PLA concentration of 9 wt. %. The optimum conditions of 1.0 ml h-1 injection rate, 25 kV electric potential and 7.0 cm needle-to-collector distance were used. A programmable KDS 100 syringe pump (KD Scientific, Holliston, Mass.) was used to transfer the polymer solution from a 1 ml syringe (Norm-Ject, Henke Sass Wolf GmbH, Germany) to the end of a 21-gauge needle (GTW-PrecisionGlide, 0.7 mm I.D., Becton-Dickinson, Franklin, N.J.). A positively charged Pt electrode of a high voltage supply (ES40P-5 W/DAM, Gamma High Voltage Research) was connected to the end of the needle. An aluminum plate connected to the ground electrode was used as the collector. To determine morphology and size, the fiber mesh was attached to a SEM stub, coated with gold using an Ion Sputter Coater (JEOL, JFC-1100) at 20 mA current for 1 min, and imaged with a JSM-5400 scanning electron microscope (SEM; JOEL, Japan) at an accelerating voltage of 10 kV. SEM images were analyzed to determine the average fiber size using the ImageJ software (National Institutes of Health, Bethesda, Md.).

### Synthesis and Characterization of Acrylamide-Terminated GRGD Peptide

The chemical structure of Ac-GRGD is shown in FIG. 1. GRGD peptide was synthesized manually on Rink Amide NovaGel™ resin in the solid phase. Briefly, the Fmoc-protected amino acid (6 eq.), DIC (6.6 eq.) and HOBt (12 eq.) were added to 100 mg resin and swelled in DMF (3 ml). Next, 0.2 ml of 0.05 M DMAP was added to the mixture and the coupling reaction was allowed to proceed for 4-6 h at 30° C. with orbital shaking. If the Kaiser test for the presence of unreacted amines was negative, the resin was treated with 20% piperidine in DMF and the next Fmoc-protected amino acid was coupled using the same procedure. To improve the reaction yield, the peptide was functionalized directly on the peptidyl resin by coupling acrylic acid to the N-terminal amine group under conditions used for the amino acid coupling reaction. The acrylamide-terminated peptide was cleaved from the resin by treating with 95% TFA/2.5% TIPS/2.5% water and precipitated in cold ether. The product was purified by preparative HPLC on a 250×10 mm, 10 μm Xterra® Prep RP18 column (Waters, Milford, Mass.), flow rate of 2 ml min-1 using a gradient 5-95% MeCN in 0.1% aqueous TFA with a photodiode array detector (model 996, Waters) at 214 nm wavelength. The retention time of the peptide was 8.04 min. The purified peptide was characterized with a Finnigan 4500 Electro Spray Ionization (ESI) spectrometer (Thermo Electron, Waltham, Mass.). In the ESI-MS spectrum, mass numbers (m/z) 457 and 479 corresponded to the monovalent hydrogen cation [(M+H)+] and monovalent sodium cation [(M+Na)+] of the purified Ac-GRGD peptide, respectively. Fabrication of fiber-reinforced laminated composites

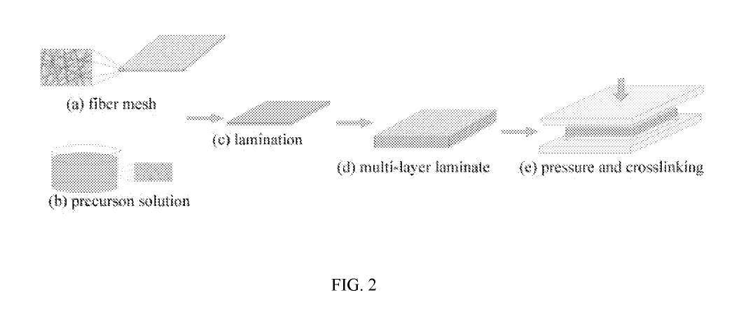

A schematic diagram of the lamination process is shown in FIG. 2. Fiber layers were coated with the hydrogel precursor solution, coated layers were stacked together and compressed, and the multi-layer assembly was crosslinked to form a laminated nanocomposite. The hydrogel/HA precursor solution was prepared by first dispersing HA nanowhiskers (principle axis=80 nm and aspect ratio=4; Berkeley Advanced Biomaterials, Berkeley, Calif.) in water. HA nanocrystals (10% by weight of PLEOF, 32 mg HA) were added to 0.825 ml PBS and the resulting dispersion was sonicated for 5 min with a 3 mm probe sonicator (Cole-Parmer Instruments, Vernon Hills, Ill.) to break down the aggregates and produce a homogeneous dispersion. Next, 30 mg of BISAM and 3.2 mg of Ac-GRGD (1% by weight of PLEOF) peptide were dissolved in the HA dispersion, followed by the addition of 315 mg of PLEOF macromer, and the mixture was heated to 50° C. and vortexed to aid dissolution. Then, 105 μl of 0.2 M APS and 105 μl of 0.2 M TMEDA were added to initiate polymerization. The L-PLA nanofiber mesh was placed on a Teflon plate and the hydrogel precursor solution was brushed over the fibers, then another nanofiber layer was placed on top of the precursor solution. This process was repeated to produce a four-layer laminated composite. The precursor solution acted as a “glue” to hold together the fibrous layers. Another Teflon plate was placed on top of the laminate, a pressure of 4.7 kPa was applied to the assembly to squeeze out the extra solution, and it was allowed to crosslink for 30 min at 50° C. The final thickness of the laminate was 85±15 μm.

### Characterization of Fiber-Reinforced Laminated Composites

The laminated composites were imaged with an SEM with accelerating voltage of 10 kV as described further herein. For determination of water uptake, crosslinked laminated disks (8 mm diameter) were soaked in PBS for 24 h at 37° C. with a change of medium every 6 h before the swollen weight, Ws, was measured. Next, the disks were placed in distilled deionized (DDI) water for at least 12 h to remove excess electrolytes. Then samples were dried under ambient conditions for 12 h, followed by drying in vacuum at 40° C. for 1 h, when the dry sample weight, Wd, was recorded. The equilibrium water uptake of the laminates was determined by Q=(Ws−Wd)/Wd. Degradation was measured as a function of time in 5 ml primary medium (DMEM supplemented with 10% FBS, 100 U ml-1 penicillin (PEN), 100 μg ml-1 streptomycin (SP), 50 μg ml-1 gentamicin sulfate (GS) and 250 ng ml-1 fungizone (FZ)), without fetal bovine serum (FBS) at 37° C. under mild agitation. At each time point, samples were removed from the medium, washed with DDI water and dried in vacuum. The dry sample weight was recorded and compared with the initial dry weight to determine fractional mass remaining A Rheometrics RSA III dynamic mechanical analyzer (DMA; TA Instruments, New Castle, Del.) was used to measure Young's modulus of the laminated nanocomposites at 37° C. The DMA was used in the static mode at a strain rate of 0.002 s-1. Rectangular films 20 mm in length, 4 mm in width and 85 μm in thickness were used to generate the strain-stress curves. BMS cell isolation and seeding

BMS cells were isolated from the bone marrow of young adult male Wistar rats. After aseptically removing the femurs and tibias, the marrow was flushed out with 20 ml of cell isolation medium (DMEM supplemented with 100 U ml-1 PEN, 100 μg ml-1 SP, 20 g m1-1 FZ and 50 μg ml-1 GS). The cell suspension was centrifuged at 200 g for 5 min and the cell pellets were resuspended in 12 ml of primary medium, aliquoted into T-75 flasks and maintained in a humidified 5% CO2 incubator at 37° C. Cultures were replaced with fresh medium at 3 and 7 days to remove haematopoietic and other unattached cells. After 10 days, cells were detached from the flasks with 0.05% trypsin-0.53 mM EDTA and seeded on laminated composites.

For cell seeding, the sample (1 cm in diameter) was placed on a sterile round glass coverslip and the edge was coated with a silicone sealant (uncatalyzed peroxide-initiated Class VI medical grade liquid silicone rubber; Dow Corning, Midland, Mich.). The sealant was allowed to harden for 12 h under sterile conditions to prevent separation of the sample from the coverslip in culture medium. Next, the construct was sterilized by ultraviolet radiation for 1 h with a BLAK-RAY 100 W mercury long wavelength (365 nm) UV lamp (UVP; Upland, Calif.) followed by 75% ethanol, and then washed with PBS prior to cell seeding. A 250 μl volume of the BMS cell suspension at a density of 4×105 cells ml-1 in primary medium was placed on each sample, which was then incubated for 24 h. After cell attachment, the medium was replaced with complete osteogenic medium (primary medium supplemented with 100 nM dexamethasone, 50 μg ml-1 ascorbic acid and 10 mM β-glycerophosphate) and cultured for up to 21 days. For imaging with SEM, cell-seeded samples were fixed with 4% paraformaldehyde (Sigma-Aldrich) in PBS for 40 min at ambient conditions. Next, samples were stained with osmium tetroxide (Sigma-Aldrich), dehydrated in sequential ethanol solutions and dried by critical point drying. The dried specimen was mounted on a stub, coated with gold (Polaron sputter coater, Quorum Technologies, New Haven, UK) and observed with a JEOL SEM at an accelerating voltage of 10 kV (JSM-6300; Tokyo, Japan).

### Osteogenic Differentiation of BMS Cells on Laminated Composites

At each time point (7, 14 and 21 days), cell-seeded laminates were washed with serum-free DMEM for 8 h to remove serum components, washed with PBS, lysed and used for measurement of DNA content, ALPase activity and calcium content. The double-stranded DNA content of the samples was determined using a Quant-it PicoGreen assay (Invitrogen, Carlsbad, Calif.) according to manufacturer's instructions. An aliquot (100 μl) of the working solution was added to 100 μl of the cell lysate and incubated for 4 min at ambient conditions. The fluorescence was measured with a Synergy HT plate reader (Bio-Tek; Winooski, Vt.) at emission and excitation wavelengths of 485 and 528 nm, respectively. Measured fluorescent intensities were correlated to cell numbers using a calibration curve constructed with BMS cells of known concentration ranging from 0 to 4×104 cells ml-1. ALPase activity was assessed using a QuantiChrom ALPase Assay Kit (BioAssay Systems, Hayward, Calif.) according to the manufacturer's instructions. A 10 μl aliquot of the sonicated cell lysate was added to 190 μl of the reagent solution containing 10 mM p-nitrophenyl phosphate and 5 mM magnesium acetate, and the absorbance was recorded at time zero and again after 4 min. ALPase activity was calculated using the equation (At=4−At=0)/(Acalibrator−AddH2O)×808, and expressed as IU 1-1. The absorbance was measured on a Synergy HT plate reader at 405 nm. The calcium content was measured using a QuantiChrom Calcium Assay Kit (BioAssay Systems) according to manufacturer's instructions. A 0.2 ml aliquot of 2 M HCl was added to 0.2 ml of the sonicated cell lysate to dissolve the calcium content of the mineralized matrix. Next, a 5 μl aliquot of the supernatant was added to 200 μl of the working solution. After incubation for 3 min, the absorbance was measured with a plate reader at 612 nm. Measured intensities were correlated to equivalent amounts of Ca2+ using a calibration curve constructed with calcium chloride solutions of known concentration ranging from 0 to 200 μg ml-1. For HA containing samples, the measured intensities at day 4 (negligible mineralization after 4 days) were used as the baseline, and subtracted from the measured intensities at days 7-21.

mRNA Analysis of BMS Cells on Laminated Composites

At each time point, total cellular RNA was isolated using TRIzol (Invitrogen, Carlsbad, Calif.) plus RNeasy Mini-Kit (Qiagen, Valencia, Calif.) according to the manufacturer's instructions. The qualitative and quantitative analysis of the RNA samples was performed with NanoDrop 2000 (Thermo Scientific, Waltham, Mass.). The obtained RNA histograms and gel images were analyzed for the intact 28S and 18S ribosomal RNA. A 1 μg quantity of the extracted total RNA was subjected to cDNA conversion using the Reverse Transcription System (Promega, Madison, Wis.). The obtained cDNA was subjected to classic polymerase chain reaction (PCR) amplification with appropriate gene specific primers and the control primer ARBP (acidic ribosomal phosphoproteins) for 35 cycles. Primers for real-time PCR (RT-qPCR) analysis were designed and selected using the Primer3 web-based software. The PCR products were analyzed by agarose gel electrophoresis with 2% ethidium bromide staining (Sigma-Aldrich). The annealing temperatures and other parameters for amplification were optimized by classical PCR and agarose gel electrophoresis. RT-qPCR was performed to analyze the differential expression of osteopontin (OP), osteocalcin (OC) and osteonectin (ON) genes with SYBR green RealMasterMix (Eppendorf, Hamburg, Germany) using a Bio-Rad iCycler machine (Bio-Rad, Hercules, Calif.) and iCycler optical interface version 2.3 software. The following forward and reverse primers were synthesized by Integrated DNA technologies (Coralville, Iowa): ON: forward 5′-ACA AGC TCC ACC TGG ACT ACA and reverse 5′-TCT TCT TCA CAC GCA GTT T; OP: forward 5′-GAC GGC CGA GGT GAT AGC TT and reverse 5′-CAT GGC TGG TCT TCC CGT TGC; OC: forward 5′-AAA GCC CAG CGA CTC T and reverse 5′-CTA AAC GGT GGT GCC ATA GAT; and ARBP: forward 5′-CGA CCT GGA AGT CCA ACT AC and reverse 5′-ATC TGC TGC ATC TGC TTG. Quantification of gene expression was based on the crossing-point threshold value (CT; number of cycles required for the RT-qPCR fluorescent signal to cross the threshold) for each sample. This was evaluated by the Relative Expression Software Tool as the average of three replicate measurements. The expression of the ARBP housekeeping gene was used as the reference and the fold difference in gene expression was normalized to that at time zero. The model of Pfaffl, which includes an RT-qPCR efficiency correction factor of the individual transcripts, was used to determine the expression ratio of the gene. The CT values were processed and analyzed for standard error and significant difference between the groups with the Q-gene software.

### Statistical Analysis

Data are expressed as means±standard deviation. Significant differences between two groups were evaluated using a two-tailed Student's t-test. A value of p<0.05 was considered statistically significant.

### Results

### Polymer Characterization

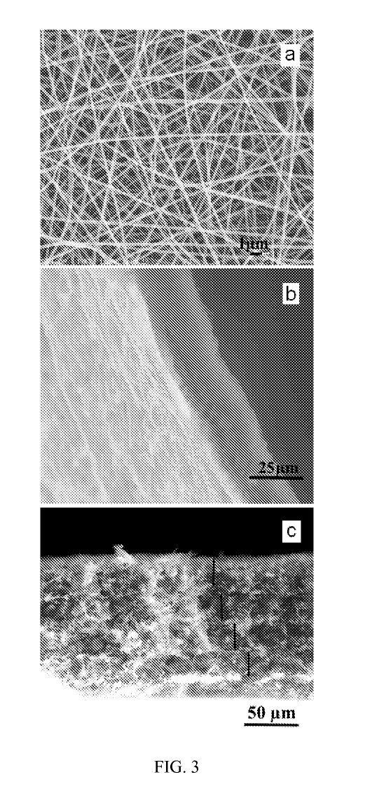

The chemical structure of the synthesized macromers was characterized by 1H NMR. The ratio of the chemical shift with peak position at 3.6 ppm to that at 5.1 ppm was directly related to the M̅ₙ̅ of LMW-PLA, which was consistent with M̅ₙ̅ values obtained from GPC. The M̅ₙ̅ and polydispersity index of LMW-PLA were 1.2 kDa and 1.4, respectively, and those of 30/70 PLEOF were 8.0 kDa and 1.6, as measured by GPC. The SEM images of the fiber mesh, one-layer laminate and four-layer laminate are shown in FIGS. 3a, b and c, respectively. The average diameter of the fibers, produced from 9 wt. % L-PLA in HFIP solvent, was 610±320 nm. The arrows in FIG. 3c show each layer in the multi-layer laminate.

### Water Uptake and Modulus of Laminated Composites

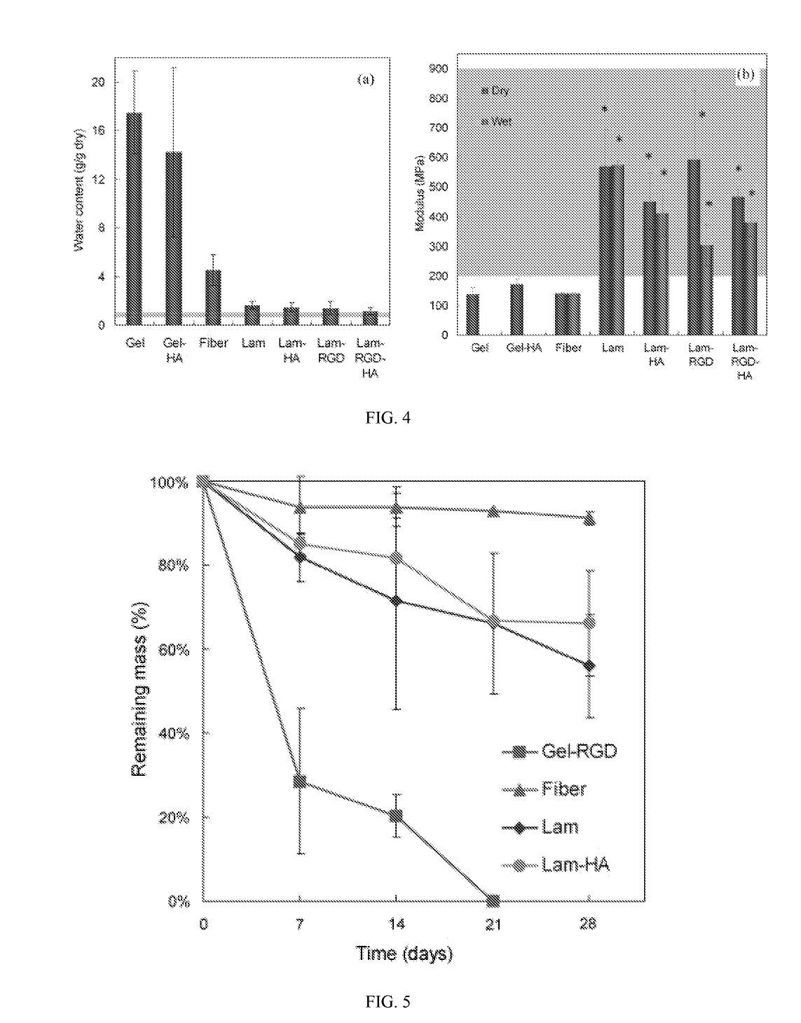

FIG. 4a shows the water uptake (weight of water/dry sample weight) of the samples. The gray area in FIG. 4a is the range for water content of the collagenous phase of the natural bone excluding the mineral component (bone water content is 15-20% by weight, corresponding to 43-50% (0.75-1.0 g g-1 dry) without the mineral component). The water uptake of the hydrogel and fiber mesh was 17.5±3.4 and 4.6±1.3, respectively. The porosity of the fiber mesh contributed to the relatively high water uptake of the hydrophobic L-PLA fibers. The addition of 10% HA to the hydrogel reduced the water content to 14.2±7.0. Lamination of the fiber mesh with the hydrogel drastically reduced the water content from 17.5±3.4 (95% water) to 1.6±0.3 (61%). For the laminates, the hydrogel swelling pressure was counterbalanced by the elastic force of the fiber mesh, resulting in a significant reduction in water uptake. With the addition of HA, RGD and RGD-HA to the hydrogel, the water uptake was reduced from 1.6±0.3 (no RGD and HA) to 1.4±0.4, 1.4±0.5 and 1.1±0.3, respectively. The higher water uptake of the ionic Ac-GRGD, conjugated to the hydrogel, was offset by the higher extent of crosslinking (via the unsaturated acrylate group of Ac-GRGD). According to FIG. 4a, lamination of the fiber mesh with the hydrogel produced composites with water content similar to that of the collagenous phase of the bone matrix.

The reduced water content of the composites with lamination affected their moduli under dry and wet conditions, as shown in FIG. 4b. The gray area in FIG. 4b is the reported range for Young's modulus of wet human cancellous bone. The modulus of the fiber mesh under dry and wet conditions was 140±3 MPa, but modulus of the hydrogel under the wet condition was two orders of magnitude lower than that under the dry condition (0.50±0.07 vs. 139±23 MPa). The addition of HA to the hydrogel did not significantly improve the modulus under the wet condition. Lamination of the fiber mesh with PLEOF hydrogel increased the modulus dramatically (compared to the fiber mesh or hydrogel) to 570±130 MPa. More importantly, Lam had a similar modulus under both dry and wet conditions (570±130 for dry and 575±14 MPa for wet). It is interesting to note that all laminated composites exhibited modulus values within the range for that of wet human cancellous bone (within the gray area), while the modulus of the fiber mesh or hydrogel was significantly less than that of cancellous bone. The fiber mesh reinforced and reduced the water uptake of the matrix, resulting in a laminated structure with robust mechanical properties under wet and dry conditions. Due to low interfacial interaction, the addition of 10% HA to the hydrogel reduced the modulus of the laminate under dry and wet conditions. It has previously been demonstrated that energetic interactions at the interface of the hydrogel and filler nanoparticles significantly affect viscoelastic properties of the composite. For example, it has been shown previously that the addition of an acrylamide-terminated glutamic acid sequence to apatite nanocrystals improves the shear modulus of the composite by an order of magnitude. The addition of Ac-GRGD to the hydrogel phase of the laminate substantially increased the modulus of the dry laminate (590±230 MPa; due to higher degree of crosslinking) but reduced its modulus under the wet condition (300±70 MPa, due to higher water uptake). The modulus of Lam-RGD-HA samples under dry and wet conditions was 470±25 and 380±47 MPa, respectively, which were between those of Lam-HA and Lam-RGD. These results demonstrate that fiber-reinforced laminated composites have the potential to provide structural support to the regenerating region in load-bearing orthopedic applications. The mechanical characteristics of the laminates can be further improved by the extent of compression in the lamination step and crosslinking, fiber to hydrogel ratio, HA content and surface treatment, water uptake of the hydrogel, fiber orientation, and number of fiber layers in the laminate.

### Degradation Characteristics of Laminated Composites

The mass loss of the L-PLA fiber mesh (Fiber), the RGD-conjugated PLEOF hydrogel (Gel-RGD), the laminate of L-PLA with PLEOF hydrogel (Lam) and the laminate with 10% (by PLEOF weight) HA (Lam-HA) with incubation time is shown in FIG. 5. The Gel-RGD sample completely degraded in 3 weeks, while the fiber mesh lost <10% of its mass in 4 weeks. The mass loss of the laminate was less than that of the hydrogel for a given degradation time, but was greater than the fiber mesh. The laminate (without HA) lost 18, 29, 34 and 44% of its mass after 1, 2, 3 and 4 weeks, respectively. The laminates with and without 10% HA showed a similar trend in mass loss with time. Hydrogel mass loss can be tailored to a particular application by varying the ratio of lactide to PEG in the macromer and extent of crosslinking For example, UV-crosslinked PLEOF hydrogels with lactide fractions of 10%, 20%, 30% and 40% had 19%, 23%, 70% and 86% mass loss after 4 weeks in primary medium at 37° C. Higher lactide fractions in PLEOF increase the density of degradable ester bonds, which increase the hydrogel degradation rate. The results in FIG. 5 demonstrate that composites with a bimodal degradation profile can be produced by varying the ratio of L-PLA fibers to PLEOF hydrogel in the laminate: a slow degradation rate by the fibers for structural stability and a fast degradation rate by the hydrogel to increase the volume for cell migration and ECM production. Adhesion of BMS cells to laminated composites

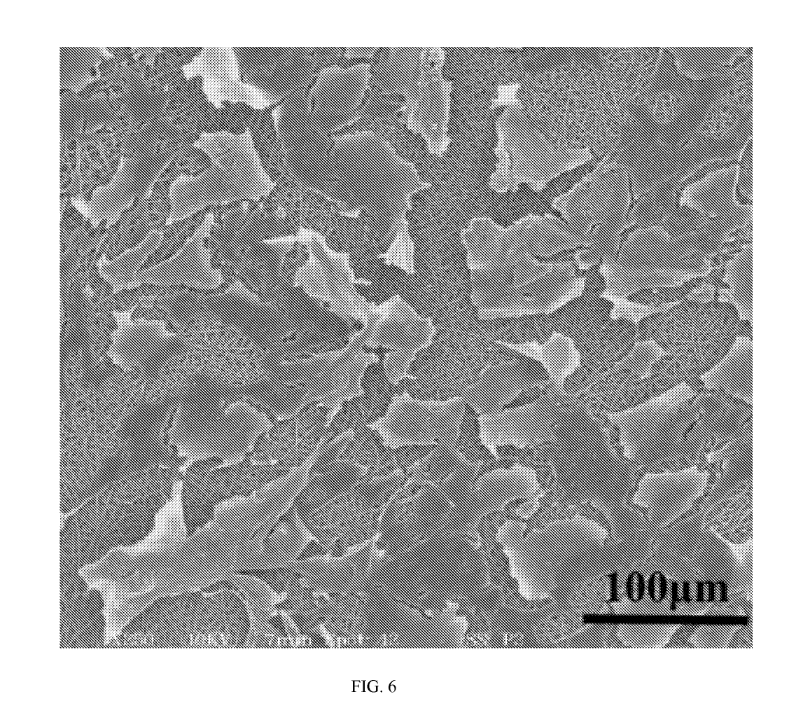

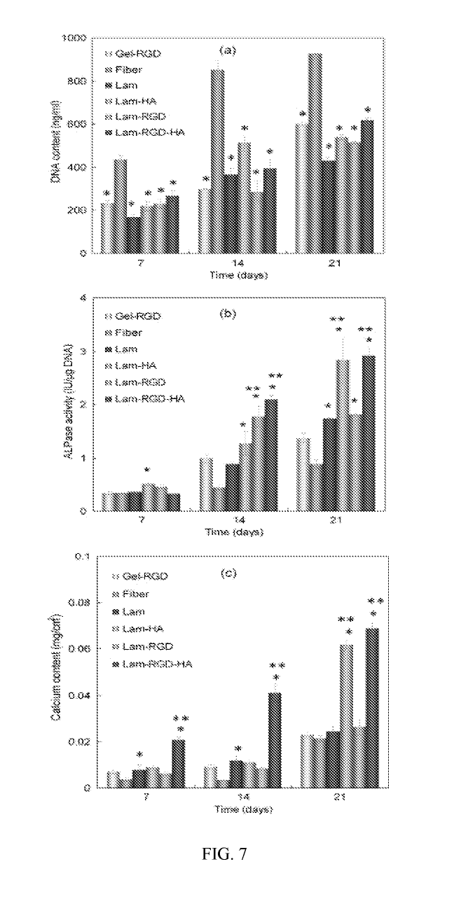

BMS cells were seeded on laminated composites and cultured in complete osteogenic medium for 21 days. Experimental groups included 30/70 PLEOF hydrogel with 1% RGD (Gel-RGD), L-PLA fiber mesh (Fiber), 4-layer L-PLA nanofiber/PLEOF hydrogel laminated composite (Lam), laminate with 10% HA (Lam-HA), laminate conjugated with 1% RGD (Lam-RGD) and laminate with 1% RGD and 10% HA (Lam-RGD-HA). A typical surface coverage and morphology of the BMS cells on a laminated surface (Lam-HA) is shown in FIG. 6. It should be noted that the morphology observed in FIG. 6 could be due to shrinkage of the cell aggregates after freeze-drying from confluent cell layers. Nevertheless, the SEM image clearly shows the cuboidal to polygonal morphology of BMS cells on the laminates. It is well established that mechanical factors such as surface hardness and modulus as well as biochemical factors affect cell adhesion and spreading. For example, it has previously been reported that fibroblast cells had a rounded morphology on untreated PLGA fiber mesh, while the cells spread on RGD-grafted fibers. The fiber mesh had relatively higher cell coverage but fewer focal point adhesions due to a lack of specific cell-substrate interactions. This was consistent with the observation that untreated electrospun poly(L-lactid-co-ε-caprolactone) (PLLA-CL) nanofibers support docking/attachment of endothelial and smooth muscle cells. Due to the non-adherent nature of the PLEOF hydrogel, the laminates without Ac-GRGD had a lower cell coverage, but that coverage was improved by reinforcement with HA, conjugation with Ac-GRGD or modification with HA and Ac-GRGD.

### Osteogenic Differentiation of BMS Cells on Laminated Composites

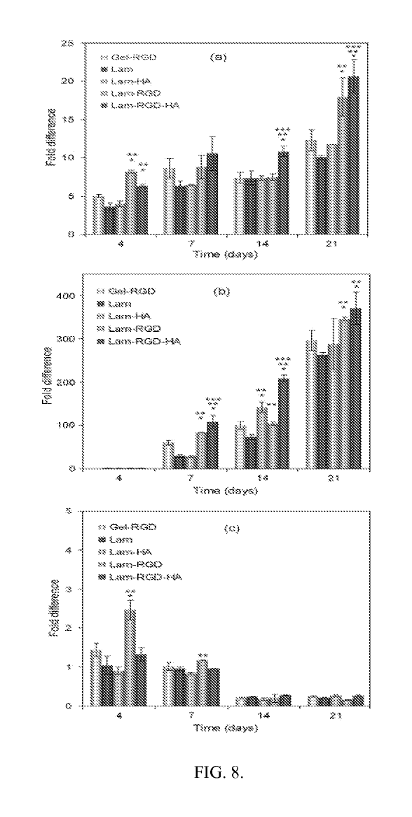

The seeded BMS cells were analyzed for DNA content, ALPase activity (early marker for osteogenesis) and calcium content (late marker for osteogenesis) with incubation time in osteogenic medium. FIG. 7a shows the DNA content of the samples as a function of time. Due to a higher surface area, the DNA content of the BMS cells on L-PLA fiber mesh was significantly higher that that of the laminate groups, as shown in FIG. 7a. However, it should be noted that osteogenic differentiation of BMS cells, which is strongly dependent on the cell-cell distance on the substrate, could be directly compared on Gel-RGD and all laminate groups because they had a similar surface area for cell seeding and similar seeding density (see FIG. 7a). Among the laminate groups, Lam-RGD-HA had the highest DNA content compared to Lam, Lam-RGD and Lam-HA for all time points. For example, the DNA content of the Lam group increased from 170±10 to 366±32 and 432±16 ng ml-1 after 7, 14 and 21 days, respectively, while that of Lam-RGD-HA increased from 267±24 to 395±43 and 620±10 ng ml-1. It has previously been observed that the normalized density of human mesenchymal stem cells on L-PLA/collagen I blend nanofibers increased from 42±8 to 60±6 and 75±5 when the L-PLA to collagen ratio was increased from 1:2 to 2:1 and 4:1. Such results demonstrate that the substrate modulus (L-PLA fraction) plays a significant role in cell attachment. It has also been demonstrated that the adhesion of preadipocytes seeded on Tyr-Ile-Gly-Ser-Arg (YIGSR)-conjugated PEG hydrogels increased from a low of 0.2 μg gel-1 for the untreated gel to a high of 0.45 μg gel-1 for YIGSR-conjugated hydrogel. Similarly, the density of fetal human osteoblasts seeded on poly(hydroxyethyl methacrylate) (pHEMA) hydrogels increased from a low of 420±50 cells cm-2 for untreated pHEMA to 1050±100 cells cm-2 for poly(L-lysine)-coated pHEMA.

FIG. 7b shows ALPase activity of the BMS cells seeded on different samples as a function of time. The L-PLA fiber mesh showed the lowest ALPase activity for any time point, despite having the highest DNA content (see FIG. 7a). The hydrophobic nature of the L-PLA fibers and the lack of specific cell-substrate interaction did not support osteogenic differentiation of BMS cells. In contrast, the RGD-conjugated PLEOF hydrogel (Gel-RGD) and L-PLA fibers laminated with RGD-conjugated hydrogel, with relatively lower cell density compared to L-PLA fibers (see FIG. 7a), had significantly higher ALPase activity. For example, the ALPase activity of BMS cells on Gel-RGD was 1.0±0.08 and 1.37±0.09 IU μg-1 DNA after 14 and 21 days, respectively, while that of L-PLA fiber was 0.44±0.04 and 0.90±0.08 IU μg-1. The ALPase activity of the fiber-reinforced hydrogel laminate (Lam) and Lam-RGD after 21 days was 1.75±0.01 and 1.80±0.01 IU μg-1, respectively, which were significantly higher than those of the fiber and Gel-RGD (0.88±0.08 and 1.37±0.09). Lamination of the fiber mesh with PLEOF hydrogel provided a less deformable substrate for cell docking and attachment, while the RGD-conjugated hydrogel facilitated cell-laminate interaction, consistent with previous results on RGD modified substrates. Furthermore, the addition of HA to the hydrogel phase of the laminate dramatically increased the ALPase activity of BMS cells. For example, the ALPase activity of Lam-RGD-HA and Lam-HA at day 21 were 2.92±0.16 and 2.83±0.43 IU μg-1 DNA, respectively, while that of Lam-RGD and Lam were 1.82±0.01 and 1.75±0.01 IU μg-1. This is consistent with previous results showing that the ALPase activity of MC3T3-E1 preosteoblast cells on poly(ε-caprolactone) (PCL)/HA was higher than that of PCL alone. Overall, the ALPase activity of Lam-RGD-HA was higher than that of the other groups for any time point.

FIG. 7c shows the calcium content of the BMS cells seeded on different samples as a function of time. The L-PLA fiber mesh, Gel-RGD, Lam and Lam-RGD groups had similar calcium contents, ranging between 0.003 and 0.008 and between 0.021 and 0.026 mg cm-2 after 14 and 21 days, respectively. The addition of HA to the laminates (Lam-HA) increased the calcium content by 2.4-fold compared to Lam, to 0.062±0.002 mg cm-2 after 21 days. RGD conjugation and HA reinforcement (Lam-RGD-HA) increased the calcium content of the laminates by 2.7-, 3.5- and 2.8-fold (compared to the Lam) after 7, 14 and 21 days, respectively.

The expression levels of the osteogenic markers OP, OC and ON as a function of time are shown in FIGS. 8a, b and c, respectively. In FIG. 8, one star indicates a statistically significant difference between the laminate groups and Gel-RGD while two stars indicate a significant difference between the test group (Lam-HA, Lam-RGD or Lam-RGD-HA) and Lam. Three stars indicate a significant difference between the Lam-RGD-HA group and Lam-HA or Lam-RGD. OP and OC expression levels increased while that of ON decreased with incubation time. At 14 and 21 days, the Lam-RGD-HA group had a significantly higher OP expression compared to Gel-RGD (one star), Lam (two star) and Lam-HA or Lam-RGD (three stars), consistent with the ALPase activities and calcium contents shown in FIGS. 7b and c, respectively. After 7 and 14 days, the Lam-RGD-HA group had a significantly higher OC expression compared to Gel-RGD (one star), Lam (two star) and Lam-HA or Lam-RGD (three stars), consistent with the ALPase activities and calcium contents shown in FIGS. 7b and c, respectively. After 21 days, the fold differences in OP expression of the Gel-RGD, Lam, Lam-HA, Lam-RGD and Lam-RGD-HA groups were 12, 10, 12, 18 and 21, respectively, and those of OC were 297, 262, 288, 346 and 371. After 21 days of incubation, the OP expression of the Lam-RGD-HA group was higher than that of Gel-RGD, Lam and Lam-HA, while the OC expression of Lam-RGD-HA was higher than that of Gel-RGD and Lam. The fold difference in ON expression decreased with time, as shown in FIG. 8c, and Lam-RGD had the lowest ON expression (0.16-fold) after 21 days. The changes in the expression level of osteogenic markers with time, shown in FIG. 8, are consistent with the reported expression levels for BMS cells on porous collagen-glycosaminoglycan scaffolds cultured in osteogenic medium. These results demonstrate that conjugation of RGD modulated the osteogenic activity of the laminated composites by enhancing cell adhesion and spreading.

The differentiation of BMS cells seeded on collagen I-coated polyacrylamide gels with a tunable degree of elasticity has been previously investigated. On soft gels with an elastic modulus <1 kPa, BMS cells exhibited a branched filopodia-rich morphology, while on stiff gels with modulus >25 kPa, which mimicked the crosslinked collagen phase of osteoids, BMS cells exhibited a polygonal morphology similar to that of osteoblasts. Furthermore, the transcription factor CBFα1 (an early marker for osteogenesis) of the BMS cells was upregulated on stiffer gels compared to softer gels. Engler et al. also showed that nonmuscle myosin II was implicated in the elasticity-directed differentiation of BMS cells to different lineages. The present results support these findings, as the ALPase activity (FIG. 7b), calcium content (FIG. 7c) and OP (FIG. 8a) and OC (FIG. 8b) expression of BMS cells were significantly higher on stiffer fiber-reinforced RGD-conjugated laminates compared to on Gel-RGD (not reinforced with L-PLA fiber mesh).

The physical and mechanical properties of the laminates and the differentiation of seeded BMS cells can be further improved by varying the fiber fraction and orientation, hydrophilicity of the hydrogel, amount, size and surface treatment of apatite nanocrystals, number of layers in the laminate and conjugation of bioactive agents. Laminated composites with macropores can be produced by the addition of a porogen (e.g. sucrose crystals, gelatin microspheres, sodium chloride crystals) to the hydrogel precursor solution followed by laminating, crosslinking and leaching the porogen from the laminate. The macroporous composites could be attractive as a scaffold in a variety of applications in regenerative medicine to provide structural support and pore volume for integration with the surrounding tissue. These include osteochondral grafts for cartilage lesions, grafts for torn tendons, cardiac patches for regeneration of myocardium after myocardial infarction and small diameter vascular grafts.

### Conclusions

Lamination of L-PLA fiber mesh with PLEOF hydrogel/HA precursor solution produced a multi-functional substrate for bone formation. The fiber layers can provide structural support and dimensional stability to prevent the regenerating region from soft tissue collapse. For example, lamination increased the modulus dramatically (compared to the fiber mesh or hydrogel) under the wet condition to 570±130 MPa, within the range for that of wet human cancellous bone, while the modulus of the fiber mesh (140±3 MPa) or hydrogel (0.50±0.07 MPa) was significantly less than that of cancellous bone. The hydrogel phase could support diffusion of oxygen and nutrients, solubilization of growth factors and conjugation of bioactive peptides, as well as providing a matrix with bimodal degradation profile: a slow degradation phase for structural stability and a fast degradation phase to increase the volume for cell migration and ECM production. For example, the laminate lost 44% of its mass after 4 weeks while the hydrogel completely degraded and the fiber mesh lost <10% of its mass in the same time. The HA nanocrystals suspended in the hydrogel phase could provide an osteoconductive substrate for differentiation and mineralization of BMS cells. For example, the BMS cells seeded on the laminates with 10% HA nanocrystals and conjugated with 1% RGD (Lam-RGD-HA group) had significantly higher ALPase activity after 14 days incubation in osteogenic medium, higher calcium content after 21 days, and higher expression of osteogenic markers osteopontin and osteocalcin after 21 days compared to those on the other laminate groups (without RGD or HA), fiber mesh or PLEOF hydrogel.

In the interests of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only, and is not intended to limit the disclosure.

What is claimed is:

1. A method for forming a laminated nanocomposite comprising:
applying a hydrogel precursor solution to a first layer of nanofiber mesh, the nanofiber mesh comprising a biocompatible synthetic polymer;
stacking a second layer of nanofiber mesh on the first layer, at least a portion of the hydrogel precursor solution being situated between the first layer and the second layer;
compressing the first layer and second layer together wherein the first layer and second layer are crosslinked to one another by the hydrogel precursor solution to form a laminated nanocomposite.

2. The method of claim 1, wherein the hydrogel precursor solution comprises poly(lactide-co-glycolide-ethylene oxide-fumarate), hydroxyapatite, or combinations thereof.

3. The method of claim 1, further comprising wrapping the laminated nanocomposite around a cylindrical rod to form a fiber-reinforced laminated tubular nanocomposite.

4. The method of claim 1, wherein the hydrogel precursor solution comprises hydroxyapatite.

5. The method of claim 1, wherein the hydrogel precursor further comprises one or more bioactive agents.

6. The method of claim 1, wherein at least one of the first layer and second layer comprise poly(L-lactide).

7. The method of claim 1, wherein the first layer is formed by electrospinning.

8. The method of claim 1, wherein the first and second layers are formed by electrospinning.

9. The method of claim 1, further comprising applying a hydrogel precursor solution to the second layer.

10. The method of claim 1, further comprising:
applying a hydrogel precursor solution to a third layer of mesh and stacking the third layer on the second layer;
compressing the first layer, second layer, and third layer together wherein the first layer, second layer, and third layer are crosslinked to one another by the hydrogel precursor solution to form a laminated nanocomposite.

11. The method of claim 1, wherein the hydrogel precursor solution is applied by dipping the first layer in a bath of hydrogel precursor solution.

12. The method of claim 1, wherein the hydrogel precursor solution is applied by spraying the first layer with the hydrogel precursor solution.

13. The method of claim 10, further comprising:
applying a hydrogel precursor solution to a fourth layer of nanofiber mesh and stacking the fourth layer on the third layer;
compressing the first layer, second layer, third layer, and fourth layer together wherein the first layer, second layer, third layer, and fourth layer are crosslinked to one another by the hydrogel precursor solution to form a laminated nanocomposite.

14. A laminated nanocomposite comprising:
a first layer of poly(L-lactide) nanofiber mesh; and
a second layer of poly(L-lactide) nanofiber mesh stacked on the first layer, wherein the first layer and second layer are compressed together and crosslinked to one another with a hydrogel precursor.

15. The laminated nanocomposite of claim 14, wherein the hydrogel precursor comprises poly(lactide-co-glycolide-ethylene oxide-fumarate).

16. The laminated nanocomposite of claim 15, wherein the hydrogel precursor further comprises hydroxyapatite.

17. The laminated nanocomposite of claim 14, wherein the first and second layers are formed by electrospinning.

18. The laminated nanocomposite of claim 14, further comprising a third layer of poly(L-lactide) nanofiber mesh stacked on the second layer, wherein the third layer and second layer are compressed together and crosslinked to one another with a hydrogel precursor.

19. The laminated nanocomposite of claim 14, further comprising a fourth layer of poly(L-lactide) nanofiber mesh stacked on the third layer, wherein the third layer and fourth layer are compressed together and crosslinked to one another with a hydrogel precursor.

20. The laminated nanocomposite of claim 14, further comprising one or more bioactive agents wherein the one or more bioactive agents comprise sucrose crystals, gelatin microspheres, sodium chloride crystals, or combinations thereof.
